(19) United States
(12) Patent Application Publication
(10) Pub. No.: US 2024/0274026 A1
(43) Pub. Date: Aug. 15, 2024

(54) MODEL OBJECT FOR VISUALLY INDICATING OVER AND UNDER REMOVALS OF MATERIAL DURING A PROCEDURE

(71) Applicant: PROMETHEAN DENTAL SYSTEMS LLC, Bogart, GA (US)

(72) Inventors: RICHARD S. CALLAN, Evans, GA (US); JERIL R. COOPER, III, Rising Fawn, GA (US)

(73) Assignee: PROMETHEAN DENTAL SYSTEMS LLC, Bogart, GA (US)

(21) Appl. No.: 18/619,562

(22) Filed: Mar. 28, 2024

Related U.S. Application Data
(63) Continuation-in-part of application No. 17/480,080, filed on Sep. 20, 2021.

(51) Int. Cl. G09B 9/00; A61B 34/10; G09B 23/28

(52) U.S. Cl. CPC G09B 9/00; A61B 34/10; G09B 23/283; A61B 2034/105; A61B 2034/107

(57) ABSTRACT

A model tooth, that is constructed with multiple layers of material. The object is designed for a particular procedure, such as removal of material to repair a cavity. The 3D area to be removed is provided in a first color, the area bordering the material to be removed is provided in a second color and the area on past the border is provided in a third color. Advantageously, upon performance of a procedure, the object can be visually inspected to determine if all of the first color material is removed, without over removing the material of the second color.

### CROSS-REFERENCES

This is a utility patent application being filed in the United States as a continuation-in-part of United States Patent Application that was filed on Sep. 20, 2021 and assigned Ser. No. 17/480,080, which application claims the benefit of United States Provisional Application for Patent filed on Sep. 20, 2020 and assigned Ser. No. 63/080,785. This application is related to the U.S. application bearing the title of “A VIRTUAL TOOL FOR DEVELOPMENT OF PSYCHOMOTOR SKILLS AND COMPARING PSYCHOMOTOR SKILL PERFORMANCE” filed on Sep. 20, 2020 and assigned Ser. No. 17/026,276. Each of the above-referenced applications are incorporated herein by reference in their entireties.

### BACKGROUND

I am sure you have heard the old adage “practice makes perfect.” Well, if you have ever tried to perfect your golf game, you know that it is not necessarily true. Sometimes practice only frustrates, costs a wad of money and confirms that yes, you are always going to be a three-digit golfer and you should never get emotionally attached to any particular ball, especially on a water course. So, sometimes you may find yourself in a situation where you have thousands of dollars of the finest equipment, countless hours logged in on the driving range and a spouse and children that never see you on the weekend, but yet, you are not anywhere close to being “the perfect golfer.”

This phenomenon presents itself in many scenarios. Wouldn't it be wonderful to walk through a scanner and have your entire being analyzed to identify what you would be good at and what you will never be good at before you invest loads of time and money into trying? There are also many careers that are plagued by this phenomenon. One such career is that of the dentist, but there are many others that similarly require more than just attaining head knowledge through education. These occupations include surgeons, artists and hairstylists, each of whom requires the accumulation of head knowledge and a certain degree of motor skills to perform their jobs proficiently, safely and reliably.

Looking further at the example of dentistry, it may not be immediately obvious that an individual lacks the ability to develop the required skills to perform the job. Much research has been conducted in the field of psychomotor skills development. Psychomotor learning is the relationship between cognitive functions and physical movement. In psychomotor learning research, attention is given to coordinated activity involving the arms, hands, fingers and feet, while verbal processes are not emphasized. Common behavioral examples include driving a car, throwing a ball and playing a musical instrument.

According to Paul Fitts and Michael Posner's three-stage model, when learning psychomotor skills, individuals progress through three stages: (1) the cognitive stage; (2) the associative stage, and (3) the autonomic stage. The cognitive stage is marked by awkward slow and choppy movements that the learner tries to control. The learner has to think about each movement before attempting it. In the associative stage, the learner spends less time thinking about every detail, however, the movements are still not a permanent part of the brain. In the autonomic stage, the learner can refine the skill through practice, but no longer needs to think about the movement as it has become a natural extension of the programming in the brain.

In view of this, one can appreciate that it may take time to fully ascertain if an individual has the ability to learn the necessary psychomotor skills. For instance, in the field of dentistry, a student may be as much as 2 to 3 years into the program before it becomes apparent that he or she will never be able to learn the required psychomotor skills to perform the job competently, if at all. Thus, similar to having a garage full of the finest golf equipment that money can buy, a student may find that he or she is hundreds of thousands of dollars in debt before realizing that the aspirations of a successful dental practice are simply unattainable.

Looking at this problem in more detail, it is most common for dental schools to employ a subjective evaluation of preclinical performance examinations. Regardless of class size and type of practical examination, significant faculty hours are involved, and the process is tedious and prone to inconsistencies. In addition, numerous studies have revealed the limitations of the subjective evaluation technique and student dissatisfaction regarding this process.

A system that can provide the type of immediate feedback that assists students in identification of preparation errors would be of tremendous value; both as a student teaching aid and as an evaluation instrument for educators. Computerized assessment models have existed in dental education for many years. The implementation and utilization of digitized technologies, although not yet ubiquitous, has slowly increased in numerous dental schools across the world. The discovery of appropriate application of these technologies continues to be an area of intense examination. This examination has led to an increased understanding of the potential of these technologies and served as an impetus for continued improvement.

Planmeca Compare software (E4D Technologies, Richardson, TX) was developed to assist students in learning proper tooth preparation skills by providing a comparison of their preparation to a master or ideal preparation. Many studies have verified the accuracy, validity, reliability and reproducibility of the comparisons obtained using the Planmeca Compare software. The Planmeca Compare software provides the user with both visual and numeric feedback that measures the degree of correlation between the tooth augmentation created by a student and that by a master or ideal preparation. The software allows the user to assign a tolerance value—e.g., 300 microns (μ)—which essentially creates a three-dimensional zone around the ideal preparation. If that tolerance level were 300μ, any under reduction (blue) or over reduction (red) of the student's preparation extending beyond that tolerance level would be reflected as a decrease in the percent comparison number recorded for the sample preparation. If, for example, 80% of the surface area of the student's sample preparation lies within this tolerance zone, it is considered to correlate at 80%. With smaller tolerance values, lower percentages of correlation are reported.

The cost of a dental education continues to rise. The 2017 average of student loan dental debt was $287,331 for the four years of dental school. Despite that fact, the number of applicants to dental schools has increased over 50% in the last ten years. In 2016, nearly 12,000 individuals applied to U.S. accredited dental schools. With a current total of 66 dental schools in the United States and Canada, gaining acceptance is still a challenging proposition.

A dental education is a long and arduous process. Many students find it difficult to overcome the inherent mental and physical challenges. The academic load is intense and the additional burden of learning and developing clinical abilities can be overwhelming to some. Dental educators strive to create the ideal curriculum and find the optimum teaching methods and modalities to help students overcome these challenges and achieve the knowledge and skills necessary to become competent practicing dentists.

The process begins with the selection of the right candidate. An applicant should possess both the didactic knowledge and the manual dexterity required to be successful, not only in dental school, but also in a dental career. In 1950, the Council on Dental Education, working with a committee from the American Dental Education Association, introduced the Dental Aptitude Test (DAT). At its inception, the DAT included a chalk-carving test that was designed to indicate the motor skills of the individual applicant. In 1972, the chalk-carving test was replaced by the Perceptual Aptitude Test (PAT). Only a few dental schools continue to use chalk carving as a criterion for entrance into their programs.

Currently, admission committees from the various dental schools rely on a combination of student's overall grade point average (GPA), science GPA, the overall Dental Aptitude Test (DAT) score and interviews to guide them in selection of students for admission into dental school. The DAT is a standardized test that includes four sections: natural sciences, reading comprehension, quantitative reasoning and perceptual ability (PAT). Unfortunately, the DAT does not provide a score or assessment regarding psychomotor skills. However, the PAT does challenge individuals in six different areas: apertures, view recognition, angle discrimination, paper folding, cube counting and 3D form development. While this is a good measure of spatial visualization skills, it does not test an individual's psychomotor skills. Without a way to test for psychomotor skills prior to dental school admission, faculty are faced with the task of trying to determine if students can learn the psychomotor skills necessary to become a competent dentist.

Identifying students that can both survive the academic workload and acquire the hand skills necessary to succeed in the clinical environment is a difficult task. Studies on correlations between admissions criteria (i.e., PAT scores) and dental school performance have produced mixed results. Lacking a valid metric by which to assess an individual's psychomotor skills adds additional uncertainty to the selection process.

Although graduation rates remain high for most, many schools are experiencing an increasing rate of student dismissals and all are evaluating ways to reduce that unfortunate outcome. Poor psychomotor skills were not identified separately from academic difficulties as a reason for dismissal, but the elevated rate has caused psychomotor skills to become a focus of attention for dental admissions.

Many times a student is not aware of a deficiency in their psychomotor skill ability. Often this deficiency can go virtually undetected until well into their dental education, making it difficult to dismiss someone who has spent so much time and so much money pursuing a potentially unattainable goal. In addition, institutions spend an inordinate amount of resources on remediating the struggling student and, unfortunately, allowing a minimally competent student to graduate. It would save the student and the school untold time and money if this issue were addressed either earlier in the curriculum or prior to admission into dental school.

Students gain psychomotor skills in many different ways. The current method of teaching and assessing motor skills is through preclinical courses that include waxing teeth and preparing plastic manikin teeth. Students receive the preparation parameters for a clinically acceptable tooth preparation from presentations (such as a POWER POINT presentation), preclinical courses and online resources. Following this instruction, dental students spend countless hours practicing tooth preparations on plastic teeth. However, proficiency simply by practice is not an absolute. If a student practices a preparation incorrectly and without proper feedback, he or she will continue to perfect the imperfection instead of learning how to do the preparation correctly. Deliberate practice, on the other hand, refers to a special type of practice that is purposeful and systematic. Deliberate practice is accomplished by focusing on critical aspects of a process and then concentrating on those specific areas in order to improve performance. During deliberate practice, a student uses cognitive ability to access previous knowledge. Tapping into this cognitive ability also teaches the student to know when, where and how to apply it in conjunction with motor skill improvement. It has been stated that skill development and expertise are strongly related to the time and efficiency of deliberate practice.

Virtual haptic simulation is fast becoming an area of interest to improve the psychomotor skills of dental students. Unlike other dental simulators that are primarily visual simulation, the haptic simulator gives the participant the sense of touch and proprioception when practicing dental preparations. Haptic simulators are reliable and can distinguish between expert and novice performance. The qualitative and quantitative feedback received from these technologies gives the student and the institution the ability to track improvement. Thus, haptic simulation provides the opportunity for students to exercise deliberate practice, all the while receiving immediate objective feedback on one's performance and growth.

A haptic simulator allows an individual to feel what it is like to hold a dental high-speed handpiece and a dental mouth mirror while preparing either a tooth or an area on a module. This simulation includes tactile sensation, giving the participant a realistic experience similar to that of working on a live patient.

Students rely on a combination of faculty feedback and self-evaluation for their progress. Without objective feedback, improving performance is difficult. Research has indicated that immediate feedback is more effective in performance gains than receiving delayed feedback. However, depending on the student-to-faculty ratio, this important feedback can sometimes be delayed. In addition, even though performance parameters are dictated, faculty assessment remains largely subjective based on their individual perception of how well the tooth preparation meets the parameters. The Planmeca Compare technology provides an accurate, valid and timely assessment of a student's performance.

The current generation has grown up in an electronic environment, and they are quite accustomed to computers and computer games. A haptic simulator can provide an easy, realistic means by which a student can exercise deliberate practice towards the purpose of improving their psychomotor skills and accomplishing specific tooth preparations at a clinically acceptable level.

It would thus be advantageous to have a system that tests, evaluates and ascertains if an individual has the propensity to learn specific psychomotor skills. The present invention as described further below, presents such a system for the field of dentistry.

### SUMMARY

Embodiments of the present invention include a model object, such as a model tooth, that is constructed with multiple layers of material. The object is designed for a particular procedure, such as removal of material to repair a cavity. The 3D area to be removed is provided in a first color, the area bordering the material to be removed is provided in a second color and the area on past the border is provided in a third color. Advantageously, upon performance of a procedure, the object can be visually inspected to determine if all of the first color material is removed, without over removing the material of the second color.

In addition, embodiments of the present invention include a system and method for enabling individuals to physically and virtually perform dental procedures on objects, such as teeth, and receive immediate performance feedback. More specifically, an exemplary embodiment includes a system to identify differences between dental preparations and/or restorations and an ideal dental preparation and/or restoration. In the exemplary embodiment, the system includes a scanning sub-system, a compare sub-system, a converter sub-system and a haptic sub-system. The scanning sub-system is configured to scan an intraoral or freestanding tooth to generate a digitized file representation of the tooth. The compare sub-system is configured to interface with the scanning sub-system to receive a model tooth digitized file. The converter sub-system is configured to interface with the compare sub-system to receive the model tooth digitized file and to convert the model tooth digitized file. As such, the model tooth digitized file is converted to a model tooth digitized file that is modified in one or more of the following manners: scaled, cropped and/or reduced in size (where scaling means to adjust the actual size of the object within the digitized files, such as changing the size of the model tooth such as by zooming in or zooming out).

The haptic sub-system is configured to receive the converted model tooth digitized file and to present a 3D rendering of the model tooth on a screen. Further, the haptic sub-system includes a hand-held tool and a tool simulator that simulates one of a plurality of dental tools, wherein each of the plurality of simulated dental tools defines a tool operation and a haptic feedback for the dental tool, and further enables a first individual to virtually perform augmentations to the model tooth and create an augmented digitized file. The converter sub-system is then further configured to receive the augmented digitized file and adjust the augmented digitized file as necessary. The compare sub-system is then further configured to receive the adjusted augmented digitized file from the converter sub-system and to compare the adjusted augmented digitized file to an ideal augmented digitized file and generate a comparison report indicating the differences between the adjusted augmented digitized file and the ideal augmented digitized file.

Further, the haptic sub-system is also configured to enable a second individual to virtually perform augmentations to the model tooth and create an ideal augmented digitized file.

These and other embodiments, features, functions and aspects of the invention are presented in the figures and the detailed description of the figures.

### BRIEF DESCRIPTION OF DRAWINGS

FIG. 1 is a block diagram illustrating an exemplary environment along with the various functional sub-systems and the logical interfaces between the sub-systems.

FIG. 2 is a flow diagram illustrating steps, actions or procedures for one inventive aspect that can be performed using the system illustrated in FIG. 1.

FIG. 3A is a simplified two-dimensional view of an object, such as a tooth, that can be augmented by the various embodiments of the system.

FIG. 3B illustrates an ideal augmentation of the tooth of FIG. 3A, such as would be prepared by an instructor.

FIG. 3C illustrates an augmentation of the model tooth of FIG. 3A, such as would be prepared by a student.

FIG. 3D is a simplified overlay of the ideal augmented tooth of FIG. 3B and the sample augmented tooth of FIG. 3C.

FIG. 3E is a 3D representation of a master or model tooth 352 that is to be subjected to a restoration procedure.

FIG. 3F is a 3D representation of a sample restoration 354, such as one that has been prepared by a student being evaluated.

FIG. 3G is a 3D representation of a performance comparison 356 of the master tooth 352 and the sample restoration 354.

FIG. 3H is a 3D representation of a master or model tooth 362 that is to be subjected to a preparation procedure.

FIG. 3I is a 3D representation of a sample preparation 364, such as one that has been prepared by a student under evaluation.

FIG. 3J is a 3D representation of a performance comparison 366 of the master tooth 362 and the sample restoration 364.

FIG. 4 is a functional block diagram of the system components that can serve as an environment or platform for the system and/or sub-systems presented for the various embodiments herein.

FIG. 5 is a two-dimensional (2D) simplified conceptual diagram of a psychomotor skill development and assessment tool.

FIG. 6 is a conceptual diagram of a two-dimensional (2D) simplified conceptual diagram of a psychomotor skill development and five-layer assessment tool.

FIG. 7A is another exemplary embodiment of an assessment tool.

FIG. 7B is a cross-sectional view of the embodiment of FIG. 7a taken at line 7b-7b.

FIG. 7C is a cross-sectional view of the embodiment of FIG. 7a taken at line 7c-7c.

FIG. 8 is yet another exemplary embodiment of an assessment tool.

FIG. 9 is yet another exemplary embodiment of an assessment tool.

FIG. 10A illustrates an actual three-dimensional assessment tool deployable in an operational system.

FIG. 10B illustrates a top-plan view of the assessment tool of FIG. 10A within the environment of a gum or tissue.

FIG. 11A illustrates an actual three-dimensional assessment tool deployable in an operational system.

FIG. 11B illustrates a top-plan view of the assessment tool of FIG. 11A within the environment of a gum or tissue.

FIG. 12 is yet another exemplary embodiment of a target model system on which a student or practitioner can strive to work.

### DETAILED DESCRIPTION

The present invention, as well as features and aspects thereof, is directed towards providing a system and method for identifying and scoring differences between a dental preparation and/or restoration and an ideal or desired preparation and/or restoration.

In the description and claims of the present application, each of the verbs “comprise,” “include” and “have,” and conjugates thereof, are used to indicate that the object or objects of the verb are not necessarily a complete listing of members, components, elements or parts of the subject or subjects of the verb. Examples are provided, and such examples, unless otherwise indicated, should always be assumed to be non-limiting examples to provide clarity of the embodiments rather than limitations on the scope of the invention.

In this application, the term sub-system is used to delineate functionality rather than absolute divisions. As such sub-systems may exist independently and communicate with other sub-systems over a network, wired connection and/or wireless connection, or may be integrated within a single system sharing processing, memory and other resources, as well as a hybrid of these configurations. Each sub-system may be any one of, or any combination of, software, hardware and/or firmware.

An objective of the present invention is to provide a system and method that enables a student or practitioner to augment a tooth, such as by creating a preparation or restoration, in a simulated haptic environment, and then to give immediate feedback to the student or practitioner as to their performance.

Systems to generate a comparison of an augmented tooth and a sample augmented tooth, such as the Planmeca Compare system are of great benefit. Further, haptic virtual simulators, such as the Simodont system can provide a beneficial environment for testing and developing skills and psychomotor skills. The combination of these two remarkable technologies advantageously provides many opportunities not possible through the individual use of either the Planmeca Compare or the Simodont or other similar systems. The present invention focuses on creating a system and method to allow comparing systems and haptic simulators to work together to provide an overall system that addresses the current needs in the art.

The various embodiments of the present invention integrate the benefits of systems that can compare two preparations and systems, thus enabling an individual to create preparations in a virtual haptic simulated system. These two technologies, integrated with each other as well as with other technologies and technical processing of digitized data files, result in the present invention-a psychomotor skills development and evaluation system (referred to herein as the “Dental System”). The goals of the various embodiments of the present invention are to decrease the learning curve of dental students regarding their psychomotor abilities and to improve their performance in the variety of tooth preparations they will perform throughout their dental careers. The unique attributes offered by the various embodiments of the present invention address the ever-increasing need for, and application of, simulation in dental education. The various embodiments provide a percent comparison between an “ideal” preparation and a student's attempt at an ideal preparation that can be applied to all types of tooth preparations. The virtual haptic technology aspects of the various embodiments of the present invention provides a student a means by which he or she can practice any given preparation a limitless number of times in a quest to achieve the desired level of competency. As was previously pointed out in the BACKGROUND section, the adage “practice makes perfect” is not actually accurate. Further, another adage “practice does not make perfect, only perfect practice makes perfect,” attributed to Vince Lombardi, is also of real concern. Virtual haptic simulators, as revolutionary as they might be, are currently incapable of adequately assessing the quality of a tooth preparation. Comparison systems, on the other hand, provide a means of assessing the accuracy of performance but lack the ability to create preparations in a virtual haptic environment. The various embodiments of the present invention provide a new and novel way to integrate such technologies.

One of the techniques utilized to merge the virtual haptic simulator and comparison technologies relies on the use of STL format for the transfer of digital images. STL files are utilized in various industries to store information about 3D models. The STL format describes only the surface geometry of a three-dimensional object without any representation of color, texture or other common model attributes. STL files are usually generated by a computer-aided design (CAD) program, as an end product of the 3D modeling process. The STL file format has been adopted and supported by many other CAD software packages and today is widely used for rapid prototyping, 3D printing and computer-aided manufacturing.

In general, the various embodiments of the Dental System operate to scan an unprepared tooth (i.e., model), such as by performing an intraoral scan or a scan of a tooth in a typodont, to create an STL file representing the scanned tooth in 3D. Further, in some embodiments, an ideal preparation can also be scanned to create an STL file for the ideal preparation. However, in other embodiments a library of ideal preparations and model teeth may be available. The STL digitized file of the unprepared tooth is then converted and downloaded into a virtual haptic simulator. Within the environment of the virtual haptic simulator, a student can practice on the model tooth to create sample preparations or, an instructor can create an ideal preparation. Once a sample or ideal preparation is constructed, an STL file representing the same in 3D can be transferred to a compare system. Within the compare system, a student's sample preparation can be compared to a model tooth, an ideal preparation and/or other sample preparations. This provides immediate feedback to the student, thus allowing the him or her to identify shortcomings or errors and immediately take action to improve. For instance, in some embodiments, the student can continue refining the same sample or the student can start over again with the model tooth. Once the desired level of competency is achieved using the Dental System, the student can then transition to a typodont for additional practice and refinement. Within the environment of a typodont, the student can scan a preparation on the typodont to create an STL file and transfer the STL file of the typodont sample to the Dental System. Here, the typodont sample can also be compared to an ideal preparation. In addition, the student can restore the preparation, scan the completed restoration and compare it to the unprepared tooth, which now acts as the “ideal” restoration. Thus, one can appreciate that the Dental System enables comparisons of preparations and restorations, which are collectively referred to herein as augmentations.

Advantageously, with the Dental System, any training facility can individually decide which teeth they want to use and what preparations they want to download into the Dental System, as well as determine what constitutes competency. Every training facility can create their own “ideal” preparation as well as their own “ideal” restoration.

Thus, the Dental System, creating an environment to utilize comparing systems and virtual haptic simulators in tandem, is a new and novel tool for dental education and psychomotor skill development. Given the current trends in dental education and the recent demands created by the COVID 19 pandemic, it is inevitable that the use of digital simulation will only increase. In addition, this process can be an answer to the “non-live” patient licensure examinations being pursued in an increasing number of states across the country.

An exemplary embodiment of the Dental System is thus operative to identify differences between a dental preparation and/or restoration and an ideal dental preparation and/or restoration. The system includes one or more sources of digitized files that contain data to render a 3D representation of a model tooth or an augmented tooth. While the various embodiments describe the use of STL files, it should be appreciated that other 3D digitized file formats may also be utilized, such as OBJ, FBX, COLLADA, 3DS, IGES, STEP and VRML/X3D, as non-limiting examples.

Further, in addition to or in lieu of STL files, it will be appreciated that other scans may also be employed. For instance, a CT scan of a patient's tooth or mouth may also be utilized. A CT or Computed tomography scan is an extremely common imaging modality in modern medicine. With advancements in technology, it is rapidly replacing many diagnostic radiographic procedures. CT scans are created using a series of x-rays, which are a form of radiation on the electromagnetic spectrum. The scanner emits x-rays towards the patient from a variety of angles—and the detectors in the scanner measure the difference between the x-rays that are absorbed by the body, and x-rays that are transmitted through the body. This is called attenuation. The amount of attenuation is determined by the density of the imaged tissue, and they are individually assigned a Hounsfield Unit or CT Number. Using this technique, the various layers of a tooth can be identified and during a simulated processing of the imaged object, different haptic feedback can be provided for the different materials in the object.

The exemplary embodiment of the Dental System also includes a virtual haptic simulator sub-system (haptic sub-system) that is enabled to present a three-dimensional rendering of the digitized file of a particular tooth on a screen. For instance, the digitized file for a model tooth can be rendered on the display for a student to modify. The haptic sub-system thus includes the visual display of the 3D rendering of the tooth as well as a hand-held device that operates as a tool simulator. The haptic sub-system may include one or more selectable tool simulators. Each such tool simulator defines the operations, and the haptic feedback, that will govern the use of the hand-held device. Thus, the hand-held device operates to simulate one of a plurality of selectable dental tools, such as a drill. Further, in some embodiments the haptic sub-system may also include a hand-held device operating as a mirror simulator. The operator can position the mirror simulator within a simulated oral space and the haptic sub-system can render on the display the position and alignment of the mirror as well as what is being reflected within the mirror. The haptic sub-system enables an individual to virtually perform preparations and/or restorations to the particular model tooth and create a particular augmented tooth digitized file.

The exemplary embodiment of the Dental System also includes a converter. The converter interfaces to the haptic-subsystem or other source of digitized files, to receive the digitized files, such as an augmented tooth, and convert the file to a desired format by cropping, resizing and/or rescaling the 3D rendering.

The exemplary embodiment of the Dental System also includes a compare sub-system. The compare sub-system interfaces with the one or more sources of digitized files to compare. For instance, the compare sub-system may receive a particular ideal augmented tooth digitized file from a scanner and then interface with the converter to receive an adjusted augmented tooth digitized file. The compare sub-system can then perform a comparison of the adjusted augmented tooth digitized file to the ideal augmented tooth digitized file and generate a comparison report indicating the differences between the adjusted particular augmented tooth digitized file and the particular ideal augmented tooth digitized file.

The various embodiments of the Dental System are quite versatile in their ability to work within a desired environment of a training entity, such as a dental school. Thus, the sources of the digitized files may include scans of intraoral or typodont teeth, library files stored within the haptic sub-system, library files stored within the compare sub-system and/or library files available from third parties accessible by the Dental System over a network or loadable into the Dental System. As a non-limiting example, an intraoral tooth can be scanned and loaded into the Dental System. An instructor or licensed dentist can then perform an augmenting procedure on the tooth and then scan the augmented tooth as an ideal augmented tooth and store the digitized file in the Dental System. A student can then utilize the haptic sub-system to create a sample augmented tooth and then compare the sample augmented tooth with the actual tooth and the ideal augmented tooth. As another non-limiting example, these same operations can be performed with a typodont. As yet another non-limiting example, a scan of an unprepared intraoral or typodont tooth can be loaded into the Dental System and an ideal augmented tooth can be generated with the haptic subsystem using the unprepared tooth. Further, a student can also work with the unprepared tooth with the haptic sub-system to create the sample augmented tooth. The sample augmented tooth can then be compared with the virtually created ideal augmented tooth. It should be appreciated that other examples are also anticipated utilizing a variety of sources and either virtually created augmented teeth or actually working intraoral or with a typodont to create the augmented teeth.

The exemplary embodiment of the Dental System thus can render a comparison of any two digitized files that are, or have been, converted to compatible formats. The compare sub-system renders the digitized files by overlaying them with each other and highlighting the differences. The differences can be presented in a descriptive format and/or a graphical format. In the graphical format, different colors can be used to represent material that should have been removed (i.e., under reduction) and/or material that should not have been removed (i.e., over reduction). Further, for restorations, different colors can be used to represent material that should not have been added (i.e., over restoration) and material that should have been added (i.e., under restoration).

Embodiments of the present invention also include a method to identify differences between dental preparations and/or restorations and an ideal dental preparation and/or restoration. An exemplary method includes the step of obtaining a model tooth digitized file and rendering a three-dimensional representation of the model tooth digitized file on a screen of a haptic sub-system. Utilizing the haptic-sub-system, a user can select one of a plurality of dental tool simulations, wherein the selected dental tool simulations defines operations and haptic feedback for a hand-held device. Further, using the haptic sub-system, the user may virtually augment the 3D representation of the model tooth using the hand-held device in accordance with the selected dental tool simulation. Once the user has completed the augmentation, an augmented tooth digitized file can be created. The augmented tooth digitized file can then be converted, such as by modifying the scale (as defined herein) of the 3D model and then sending it to a compare sub-system.

The compare sub-system may then compare the converted augmented tooth digitized file to an ideal augmented tooth digitized file having the scaling characteristics. The compare sub-system then generates a comparison report indicating the differences between the converted augmented tooth digitized file and the ideal augmented tooth digitized file and/or attributes a score to the performance (i.e., such as a percentage deviation or a percentage of commonality).

Further, the exemplary method may perform the action of obtaining the model tooth digitized file by retrieving the model tooth digitized file from a library of a plurality of model tooth digitized files. In another embodiment, the exemplary method may perform the action of obtaining the model tooth digitized file by scanning an intraoral or free-standing model tooth to create a raw model tooth digitized file and then converting the raw model tooth digitized file to the model tooth digitized file by modifying the raw model tooth digitized file in one or more of the following manners: scaling, cropping and/or reducing a size of the raw model tooth digitized file.

Further, in some embodiments, the exemplary method may perform the action of comparing the converted augmented tooth digitized file to an ideal augmented tooth digitized file by virtually augmenting the 3D representation of the model tooth using the hand-held device in accordance with the selected dental tool simulation, creating an ideal tooth digitized file and then converting the ideal augmented tooth digitized file if necessary. It should be understood that when embodiments are described as “converting the digitized file” if necessary, this can encompass many adjustments or modifications of the file that may be necessary for the targeted system to process the file. Most typically, the conversion may include adjusting the dimensional size of the object within the digitized file so that it can be properly overlaid on an object to which it is being compared. It should also be appreciated that this conversion may take place when a user opens the digitized file, either manually or automatically.

In other embodiments, the exemplary method may perform the action of comparing the converted augmented tooth digitized file to an ideal augmented tooth digitized file by physically augmenting the model tooth using an actual physical dental tool to create an ideal augmented tooth, scanning the ideal augmented model tooth to create a raw ideal augmented tooth digitized file and creating an ideal tooth digitized file. If necessary, the augmented tooth digitized file can be converted to be compatible for the comparison.

The various embodiments may generate a comparison report indicating the differences between the converted augmented tooth digitized file and the ideal augmented tooth digitized file by rendering a 3D representation of the converted augmented tooth digitized file on a display screen, rendering a 3D representation of the ideal augmented tooth digitized file on the display screen and then overlaying the 3D representations of the converted augmented tooth digitized file and the ideal augmented tooth digitized file on the display screen. Further, the various embodiments may highlight the differences between the 3D representations of the converted augmented tooth digitized file and the ideal augmented tooth digitized file on the display screen.

In some embodiments, the method may perform the action of highlighting the differences between the 3D representations of the converted augmented tooth digitized file and the ideal augmented tooth digitized file on the display screen by presenting surfaces appearing in the converted augmented tooth digitized file that are not present in the ideal augmented tooth digitized file in a first color.

Further, in some embodiments, the method may perform the action of highlighting the differences between the 3D representations of the converted augmented tooth digitized file and the ideal augmented tooth digitized file on the display screen by presenting surfaces not appearing in the converted augmented tooth digitized file that are present in the ideal augmented tooth digitized file in a second color.

It should be appreciated that, while the various embodiments of the present invention are described within the context of augmenting teeth, embodiments of the invention may be adapted for utilization on objects other than teeth. For instance, the various embodiments of the Dental System could also be adapted for use in gum repair, reconstructive surgery, both oral or otherwise, as well as other surgical procedures. Further, embodiments of the Dental System can also be utilized for other physical and/or mechanical augmentation, such as machining a part, creating replacement parts, etc. Even further, embodiments of the Dental System may be utilized for a variety of types of psychomotor skill development and assessment.

Turning now to the figures, the various embodiments, features and aspects thereof are described in further detail.

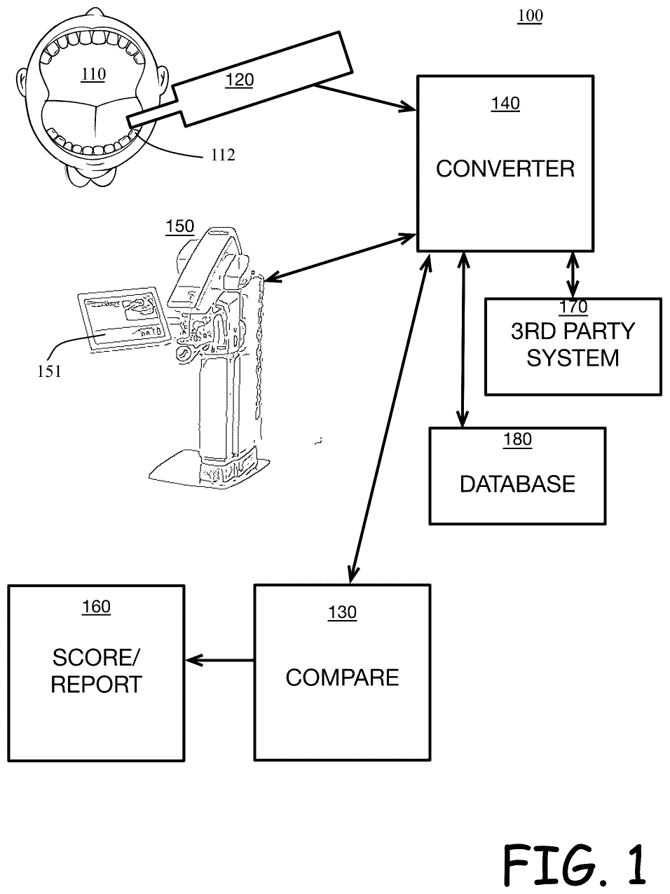

FIG. 1 is a block diagram illustrating an exemplary environment along with the various functional sub-systems and the logical interfaces between the sub-systems. Again, as previously presented, one or more of the sub-systems may be stand-alone subsystems with actual communication interfaces with other sub-systems or, one or more sub-systems may be fully or partially integrated into the same system and the interfaces may be logical interfaces.

In the illustrated embodiment of the Dental System 100, the system is shown as including a compare sub-system 130, a converter 140, a haptic sub-system 150 and a scanner sub-system 120. An exemplary Dental System 100 may also include an interface to a third-party system 170 to receive digitized files or to provide digitized files. The third-party system 170 may be another implementation of a Dental System 100 and thus, digitized files may be shared by multiple embodiments, or it may be a standard library accessible via a network, interface to a database, etc. In addition, embodiments of the Dental System 100 may interface to a database 180 for storing and retrieving digitized files.

Further, the Dental System 100 is illustrated as obtaining intraoral scans from a subject 110, but it should be appreciated that the scans can be performed on a typodont, a model, an impression or a free-standing tooth (all collectively referred to as a free-standing tooth).

Finally, the Dental System 100 also includes a score or reporting sub-system 160 that presents the results of a comparison or presents a score or ranking of a user performance. It should be appreciated that the scoring and reporting can be provided on a display screen, a print-out or encompassed in a sharable file such as a PDF, CSV, etc. format.

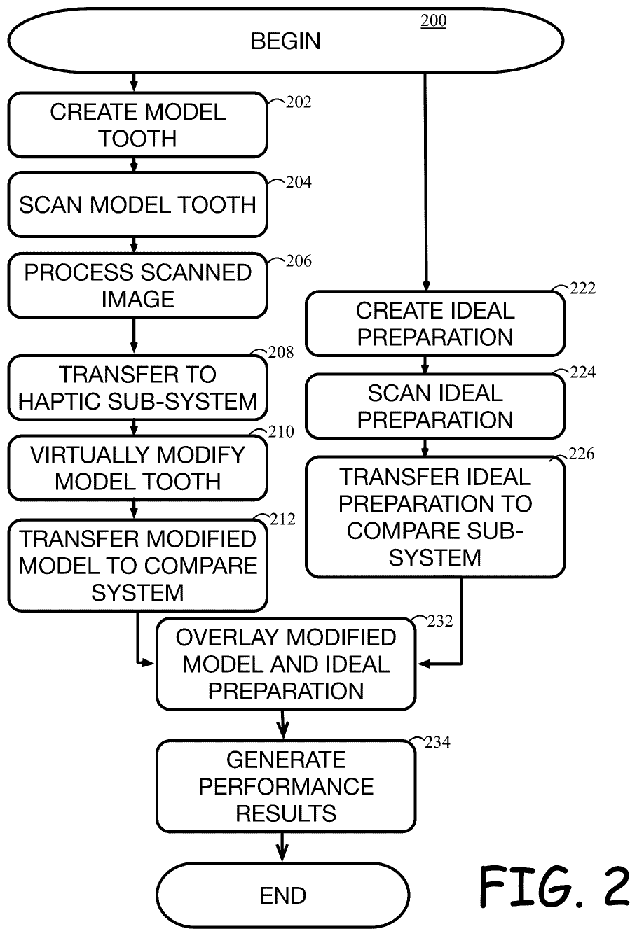

FIG. 2 is a flow diagram illustrating steps, actions or procedures for one inventive aspect that can be performed using the system illustrated in FIG. 1. The procedure 200 begins by creating a model tooth of an unprepared tooth or a previously prepared tooth that requires additional work 202. The process of creating the model tooth can be accomplished in a variety of manners such as by using an actual tooth of a patient, utilizing model teeth created from an impression of a patient's tooth, utilizing commercially available model teeth that can be purchased for installation in a typodont or even simply utilizing an object of any particular shape that is chosen to facilitate testing or development of psychomotor skills as a few non-limiting examples.

Once the model tooth or object is selected, the ultimate goal is to obtain a digitized file that defines a 3D representation of the tooth or object. The digitized file for the model tooth can be obtained in a variety of manners. In the illustrated example, the tooth is scanned 204 utilizing a scanning wand 120. As the tooth is scanned, the scanning wand 120 provides data to the system, such as converter 140, which can convert the scanned file, if necessary, to a compatible format and then provide to the compare sub-system 130, which creates and stores the digitized file. In some embodiments, the digitized file can be created by the scanning wand 120 during the scan and then transferred directly to the Dental System by sending to the compare sub-system 130, a database 180, a third-party system 170, the converter 140 and/or the haptic sub-system 150. In some applications of the Dental System, rather than scanning a tooth or object, an already created digitized file can be obtained from the compare sub-system 130, the database 180, the third-party system 170, the converter 140 and/or the haptic sub-system 150.

Compatibility with the operations that can be conducted using the embodiments of the Dental System, may rely on processing the digitized file. For example, the haptic sub-system 150 may require certain file-size constraints and adjustments to be imposed on the digital files. In such situations, the converter 140 can be utilized to reduce or enlarge the file size before transferring the digitized file to the haptic sub-system 150. Further, digitized files obtained from other sources, such as the third-party system 170 or models previously created and stored within the haptic sub-system, may have a different scaling requirement. In such situations, the converter 140 can also modify the image size of the object represented within the digitized file (or on the screen) prior to sending it to the haptic sub-system to ensure that it is compatible with and can be overlaid with other images. As a non-limiting embodiment, the converter 140 can be a digitized file editor, such as an STL editor. In an exemplary operation, the editor can be used to open a digitized file and display the 3D rendering. An operator can then crop the file to help reduce the size of the file. Further, the file can be saved with higher or lower resolution to either increase or decrease the file size respectively. The editor can also be utilized to adjust the physical size of the image embedded within the digitized file, either on screen or while saving the file. In other embodiments, the converter 140 may be fully automated and simply operate as a filter for each digitized file that passes through the converter 140 while being sent to the haptic sub-system 150, the compare sub-system 130 or other destinations. Simply by knowing the source and the destination, the converter 140 may automatically perform one or more conversions or adjustments on the digitized file.

Once a digitized file for a model tooth has been obtained and converted, the digitized file can be transferred 208 to the haptic sub-system 150. If the file is correctly formatted and accepted by the haptic sub-system 150, a user can load the digitized file onto the display of the haptic sub-system and begin to virtually modify the model tooth 210. It may be appreciated that digitized files may already be present within the haptic-subsystem 150 and simply selected by the user. The user can select which tool that the hand-held device is to simulate and begin to augment the model tooth by virtually removing or adding materials to the model tooth. Advantageously, in some embodiments the user can reverse actions that were taken to revert the model tooth back one or more operations or the user can simply reload the model tooth and start over. During operation, a user can swap out tool simulators to conduct different procedures. Once the user has completed augmenting the model tooth, the digitized file representing the augmented tooth can be saved to the haptic sub-system and/or transferred to another component or functional system within the Dental System 212. Typically, at this point, the digitized file of the augmented or modified model tooth is sent to the compare sub-system 130 through the converter 140.

Once a digitized file for an augmented tooth is received into the compare sub-system 130, the compare sub-system can perform a comparison with another digitized file, such as an ideal preparation of the original model tooth or the original tooth. The flow diagram illustrated in FIG. 2 focuses on the creation of the ideal preparation 222. The ideal preparation, as previously described, can be created in a variety of manners including using the haptic sub-system 150, physically creating the preparation and scanning it using the scanning sub-system 120, or obtaining the digitized file from a database 180, third-party system 170 or memory. If the ideal preparation is physically created, the ideal preparation is scanned with the scanner sub-system 120 to create a digitized file 226. The digitized file of the ideal preparation is then transferred 226 to the compare sub-system 130.

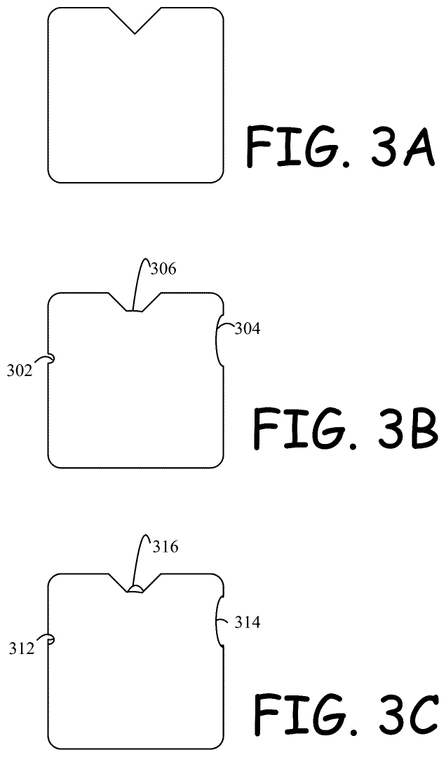

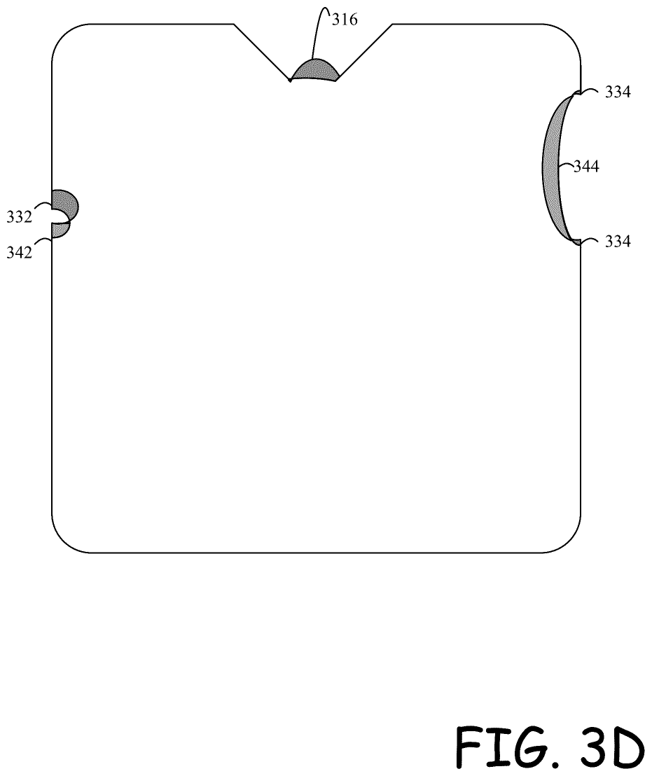

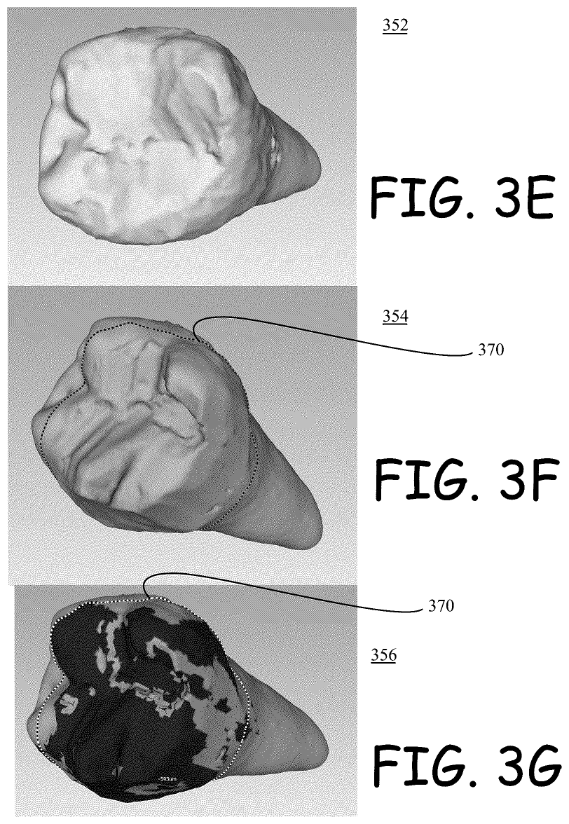

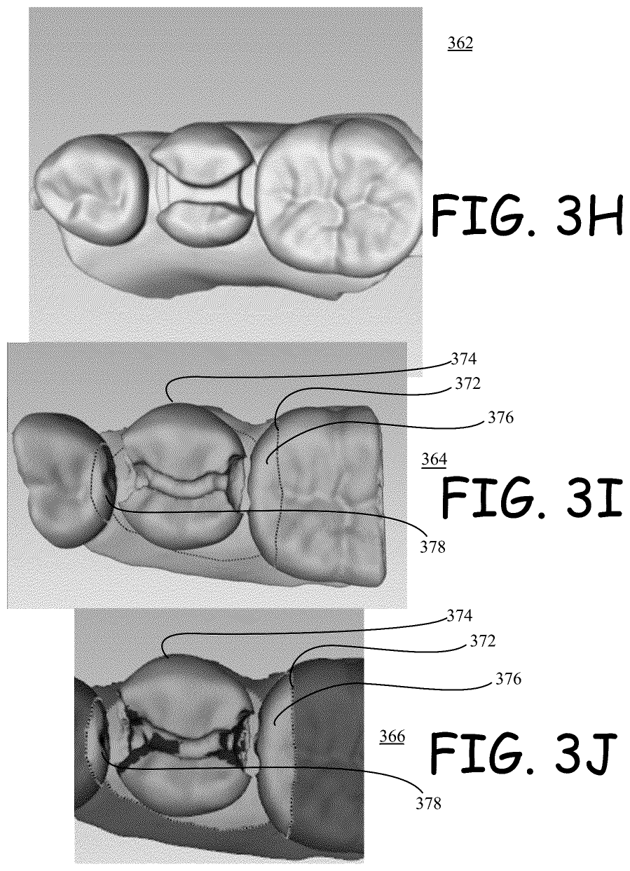

Once two digitized files to be compared are received into the compare sub-system 130, the compare sub-system 130 can overlay the two digitized files in a 3D rendering 232. The overlaid renderings can be used to show the performance of a student in creating an augmented tooth in view of an ideal tooth or the original tooth. FIG. 3A is a simplified two-dimensional view of an object, such as a tooth, that can be augmented by the various embodiments of the Dental System. The illustrated shape is assumed to be a model tooth that has not been augmented. FIG. 3B illustrates an ideal augmentation of the tooth of FIG. 3A, such as would be prepared by an instructor. FIG. 3C illustrates an augmentation of the model tooth of FIG. 3A, such as would be prepared by a student. As can be seen from FIG. 3B, a preparation has been made by removing a portion of the tooth at point 302 and a large portion at point 304. In addition, material has also been added to the model tooth at point 306. Similarly, the model tooth has been augmented to create a sample by removing a portion of the tooth at point 312 and a larger portion at point 314. In addition, material has also been added to the model tooth at point 316.

FIG. 3D is a simplified overlay of the ideal augmented tooth of FIG. 3B and the sample augmented tooth of FIG. 3C. From examining the rendering in FIG. 3D, the color blue is used to indicate under removal or over restorations and the color red is used to indicated over removal and under restorations. Thus, a student can easily look at the rendering to see material that is present that should not be (blue) and material that is missing but should not be (red). Thus, it can be seen that the student drilled an area too shallow and too low, thus creating area 332 that should have been removed and area 342 that should not have been removed. Further, the student drilled too deeply and too narrowly to create area 334 that should have been removed and area 344 that should not have been removed.

FIGS. 3E-3G illustrate a 3D representation of a model tooth restoration. FIG. 3E is a 3D representation of a master or model tooth 352 that is to be subjected to a restoration procedure. FIG. 3F is a 3D representation of a sample restoration 354, such as one that has been prepared by a student being evaluated. The compare sub-system 130 can then make a comparison of the master tooth 352 and the sample restoration 354 to generate a performance comparison. The broken line 370, as illustrated in FIG. 3F and FIG. 3G, defines the boundaries for the comparison. As such, the compare sub-system 130, in an exemplary embodiment can limit the comparison to the selected area. FIG. 3G is a 3D representation of a performance comparison 356 of the master tooth 352 and the sample restoration 354. As can be seen in FIG. 3G, the red indicates areas that are under restored and the blue indicates areas that are over restored. In various models presented herein, the assessment tool utilizes color coded materials to provide visual feedback of material that is to be removed. Likewise, color coded material or surfaces may be used to indicate areas at which restorations are to be performed. Thus, the restoration must insure that the entire color coded area is covered.

FIGS. 3H-3J illustrate a 3D representation of a model tooth preparation. FIG. 3H is a 3D representation of a master or model tooth 362 that is to be subjected to a preparation procedure. FIG. 3I is a 3D representation of a sample preparation 364, such as one that has been prepared by a student under evaluation. The compare sub-system 130 can then make a comparison of the master tooth 362 and the sample preparation 364 to generate a performance comparison. FIG. 3J is a 3D representation of a performance comparison 366 of the master tooth 362 and the sample restoration 364. As can be seen from FIG. 3J, the red indicates areas that should not have been removed (over reduction) and the blue indicates areas that should have been removed (under reduction). The broken line 372, as illustrated in FIG. 3I and FIG. 3J, defines the boundaries for the comparison. As such, the compare sub-system 130, in an exemplary embodiment, can limit the comparison to the area within the boundary. Further, in embodiments that utilize a boundary to define the compare area, the file size of the comparison and the amount of processing required to perform the comparison can be reduced. Further, irrelevant portions of a model or preparation, which may differ from each other, can be excluded from the comparison operation. In FIGS. 3F and 3G, the boundary line 370 is illustrated as including only a portion of a tooth. In such an embodiment, only the area within the boundary is compared. Thus, the student or practitioner can work on the area within the boundary and upon concluding the operation, this are can be compared to the ideal preparation. For FIG. 3I and FIG. 3J, the boundary is illustrated as including the entire tooth 374 as well as bordering or adjacent portions of neighboring teeth 376 and 378. In such an embodiment, if the student or practitioner erroneously augments one of the neighboring teeth 376 and/or 378, when the compare sub-system 130 compares the master or model tooth 362 and the sample preparation 364, this error would be detected and shown in the performance comparison 366.

The compare sub-system 130 may then generate performance results and display the results for the user (i.e., student and/or instructor) 234. The performance results may be a score that is based on the percentage of over removal, under removal, over restoration and under restoration in view of the amount of material that was intended to be removed and restored. The score can take into consideration pre-programmed tolerances. For instance, for a beginner or novice, a larger tolerance may be imposed and for an advanced student or practitioner, a smaller tolerance may be imposed.

Thus, it can be appreciated that the various embodiments of the Dental System create a new, novel and unique system and method for identifying the performance of psychomotor skills. The system can be used for a variety of applications including dental school admittance, exercise and development of psychomotor skills, licensure examinations, recertification of practitioners, dental school curriculum, etc.

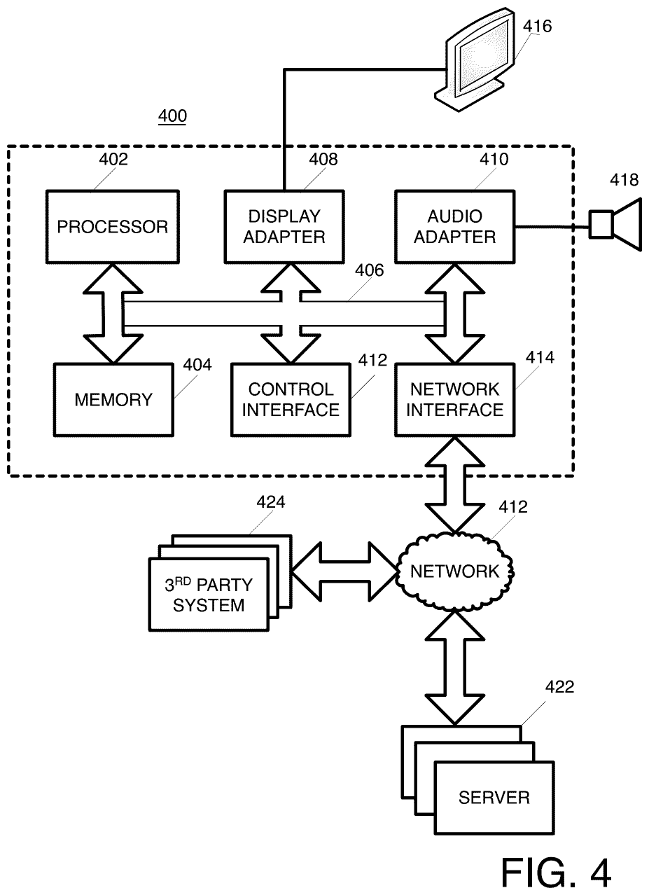

FIG. 4 is a functional block diagram of the system components that can serve as an environment or platform for the system and/or sub-systems presented for the various embodiments herein. The platform includes a controller or processor 400 that could be used in various embodiments of the disclosure for controlling aspects of the various embodiments. It will be appreciated that not all of the components illustrated in FIG. 4 are required in all embodiments of the Dental System or sub-systems thereof, but each of the components are presented and described in conjunction with FIG. 4 to provide a complete and overall understanding of the components and exemplary platform. The platform can include a general computing platform 400 illustrated as including a processor/memory device 402/404 that may be integrated with each other or communicatively connected over a bus or similar interface 406. The processor 402 can be a variety of processor types including microprocessors, micro-controllers, programmable arrays, custom IC's, discrete hardware, etc. and may also include single or multiple processors with or without accelerators or the like. The memory element 404 may include a variety of structures, including but not limited to RAM, ROM, magnetic media, optical media, bubble memory, FLASH memory, EPROM, EEPROM, disk drives, etc. The processor 402 or other components in the controller may also provide components such as a real-time clock, analog to digital converters, digital to analog converters, etc. The processor 402 also interfaces to a variety of elements including a control interface 412, a display adapter 408, an audio adapter 410 and network/device interface 414. The control interface 412 provides an interface to external controls, such as sensors, actuators, drawing heads, nozzles, cartridges, pressure actuators, leading mechanism, drums, step motors, a keyboard, a mouse, a pin pad, an audio-activated device, as well as a variety of the many other available input and output devices or another computer or processing device or the like. The display adapter 408 can be used to drive a variety of alert elements 416, such as display devices including an LED display, LCD display, one or more LEDs or other display devices. The audio adapter 410 interfaces to and drives another alert element 418, such as a speaker or speaker system, buzzer, bell, etc. The network/interface 414 may interface to a network 420, which may be any type of network including, but not limited to, the Internet, a global network, a wide area network, a local area network, a wired network, a wireless network or any other network type including hybrids. Through the network 420, or even directly, the controller 400 can interface to other devices or computing platforms such as one or more servers 422 and/or third party systems 424. A battery or power source provides power for the controller 400.

It should be appreciated that the platform presented in FIG. 4 may be a stand-alone server or may be one of several servers integrated with each other either directly or over a network. The embodiments of the Dental System may thus be implemented on a single server system or multiple server systems operating together.

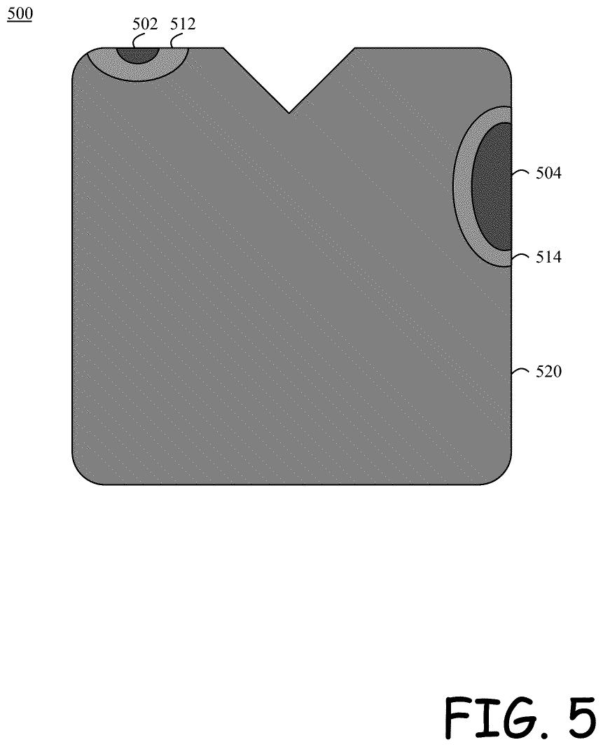

FIG. 5 is a two-dimensional (2D) simplified conceptual diagram of a psychomotor skill development and assessment tool. The assessment provides a visual and automatic feedback indicator regarding the performance of an individual related to a particular procedure or a plurality of procedures. In general, the assessment tool is an object that is constructed out of multiple layers of different colored material. The goal of the assessment tool is to provide visual guidance on how to perform a procedure and a visual indication as to the accuracy of an individual's performance of the procedure. In an exemplary embodiment, an object may include two colors of material. In such an embodiment, the material in a first color indicates material that is to be removed and material in a second color indicates material that is not to be removed. Within a dental setting, the embodiment of the assessment tool identifies material to be drilled from the tooth and material to remain.

In another exemplary embodiment, the assessment tool may be constructed from three different colors of material. Thus, as illustrated in FIG. 5, areas of different colored layers are created for the assessment tool. The embodiment illustrated in FIG. 5 is an exemplary example of a three-color material assessment tool. While the embodiment illustrated in FIG. 5 is represented in 2D, those skilled in the art will understand that the illustrated techniques can easily be adapted and applied to a 3D object. The illustrated embodiment includes an object 500 that is to be augmented by removing portions of the object. As a non-limiting example, if the object 500 is a tooth, portions of the object are to be removed by using a dental tool, such as a high-speed drill. In the illustrated embodiment, the object 500 is illustrated as including three distinct regions: (1) material to be removed during the performance of a procedure—this material is identified by the color red and includes elements 502 and 504; (2) border material that should not be removed during the procedure—this material is identified by the color green and includes elements 512 and 514; and (3) material that indicates a considerable error in conducting the procedure if it is removed—this material is illustrated by the color tan and includes element 520. Thus, with the three-color assessment tool embodiment, the border material provides a visual indicator of when to stop removing material. At the conclusion of performing a procedure, the assessment tool (for the illustrated embodiment) should optimally be free of all red colored material with only green material being visible in its place. Further, optimally no tan material will be visible through the green material. If at the conclusion of the procedure, the assessment tool includes red material, then the user has under removed material. If the assessment tool includes any tan material visible through the green material, then the user has over removed material. The thickness of the green material is, in essence, the margin of error. As such, for beginners, the green material may be thicker than for advanced individuals.

It should be appreciated that in some embodiments, the layers may be fabricated from the same material but simply be dyed or stained different colors. In other embodiments, different material may be used for the different layers. As a non-limiting example, to facilitate initial training of a beginner, the border material 512 and 514 may be selected to be a harder material so as to prevent over drilling. In other embodiments, the border material 512 and 514 may be selected to be considerably softer than the material to be removed 502 and 504, so as to better reveal errors due to over drilling.

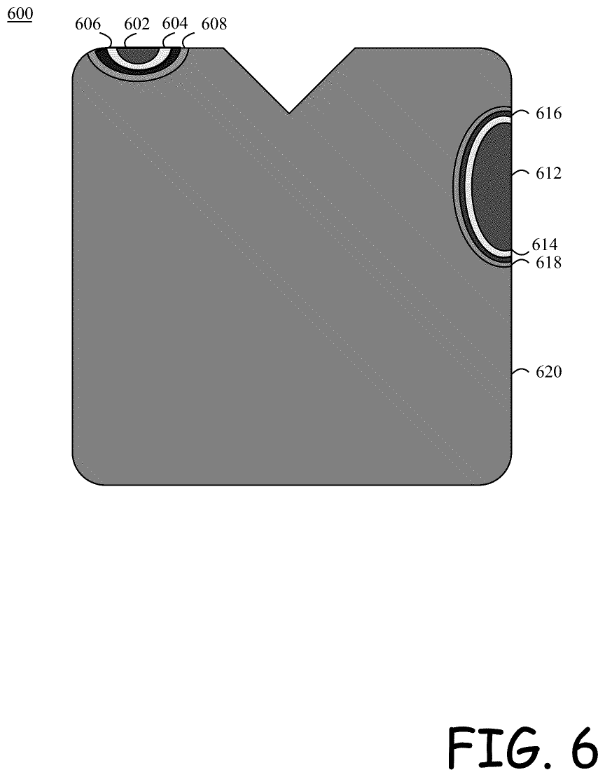

In some embodiments, additional colors may be utilized to add further granularity to the precision of the procedure. FIG. 6 is a conceptual diagram of a two-dimensional (2D) simplified conceptual diagram of a psychomotor skill development and five-layer assessment tool. This embodiment is useful to show more granularity in procedural errors. Thus, in the illustrated embodiment, if a user performing the procedure removes all of the red color 602 and 612, but leaves the yellow border 604 and 614, the user has achieved a high-level of performance. However, any blue that shows through, such as band 606 and 616 will indicate a lower level of performance. Likewise, any green that shows through, such as band 608 and 618 will indicate even a lower level of performance. And finally, any tan that shows through, such as layer 520, will indicate even a lower level of performance.

The embodiments of the assessment tool illustrated in FIGS. 5 and 6 can be actual physical models, such as a tooth in a typodont, or a virtual model. The assessment tool of FIG. 5 can be prepared in a variety of manners. As a non-limiting example, a physical embodiment of the assessment tool 500, such as would be utilized in a typodont, can be prepared in two stages. In the first stage, a procedure is performed on a model typodont tooth to over remove material from the typodont in the areas where a preparation calls for the removal of some material. The material of the typodont tooth may be a first color, such as tan. The first stage of the assessment tool is thus created by removing more material than what is desired for an ideal preparation. Next, a thin border layer of a specifically colored material can be placed over the surface of the area that was removed by the procedure. For instance, a green colored layer. Next another layer of material is laid over the thin border layer to build or restore the typodont tooth back to its original shape. This final layer may be another specific color, such as red. At this point, once the material hardens, the typodont tooth can be utilized for practicing a procedure. The procedure involves removing of the red material and not removing any of the green or tan material.

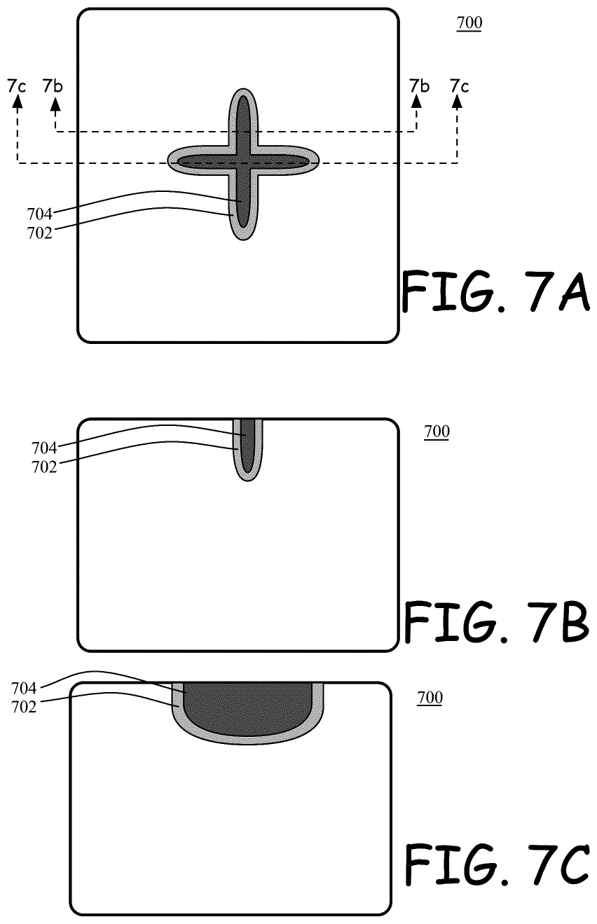

FIG. 7A is another exemplary embodiment of an assessment tool. FIG. 7B is a cross-sectional view of the embodiment of FIG. 7A taken at line 7b-7b. FIG. 7C is a cross-sectional view of the embodiment of FIG. 7A taken at line 7C-7C. In the illustrated embodiment, a block 700 includes an area to be removed 704 (illustrated in the color red) and a border area 702 (illustrated in the color green). The goal of a student or practitioner is to utilize a drill or tool to remove all of the red material without removing any of the green material. As described before, this embodiment of the assessment tool may be a virtual tool utilized fully within the Dental System or it may actually be a physical model that can be scanned into the Dental System for comparison and evaluation purposes.

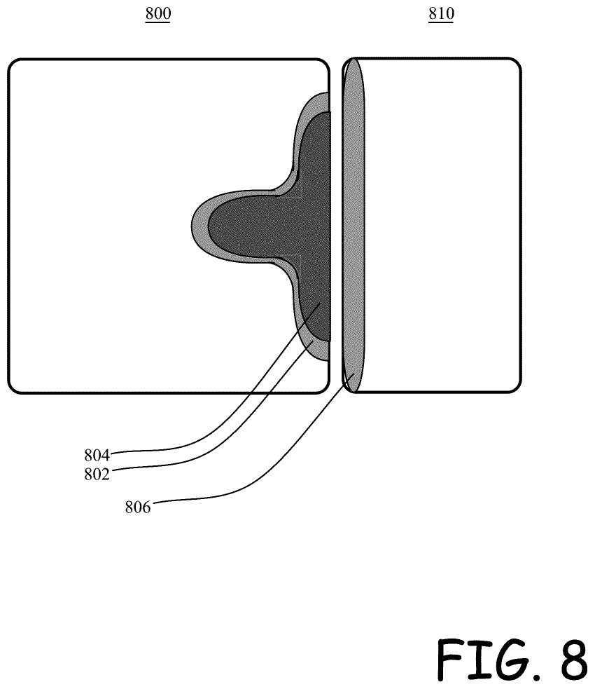

FIG. 8 is yet another exemplary embodiment of an assessment tool. In this embodiment, two blocks 800 and 810 are positioned adjacent to each other. Block 800 includes a red area 804 that is to be removed and a green border area 802 that is not to be removed. In addition, block 810 includes a green border area 806 that also is not intended to be removed. In this embodiment, a student or practitioner can practice the skills of removing material from one block without adversely impacting an adjacent block. As described before, this embodiment of the assessment tool may be a virtual tool utilized fully within the Dental System or it may actually be a physical model that can be scanned into the Dental System for comparison and evaluation purposes.

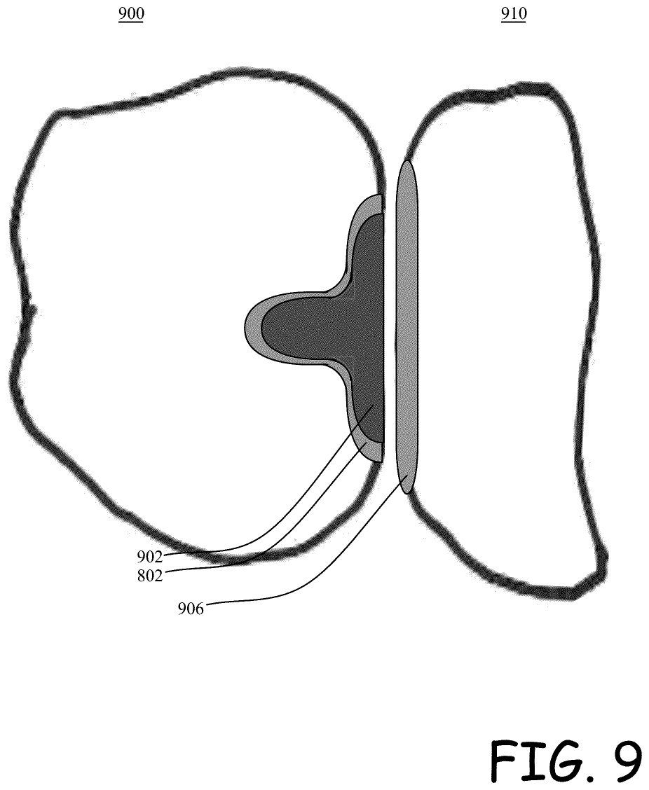

FIG. 9 is yet another exemplary embodiment of an assessment tool. In this embodiment, rather than two blocks (i.e., blocks 800 and 810 in FIG. 8), two model teeth 900 and 910 are positioned adjacent to each other. Model tooth 900 includes a red area 904 that is to be removed and a green border area 902 that is not to be removed. In addition, adjacent tooth 910 includes a green border area 906 that also is not intended to be removed. In this embodiment, a student or practitioner can practice the skills of removing material from one block without adversely impacting an adjacent block. As described before, this embodiment of the assessment tool may be a virtual tool utilized fully within the Dental System or it may actually be a physical model that can be scanned into the Dental System for comparison and evaluation purposes.

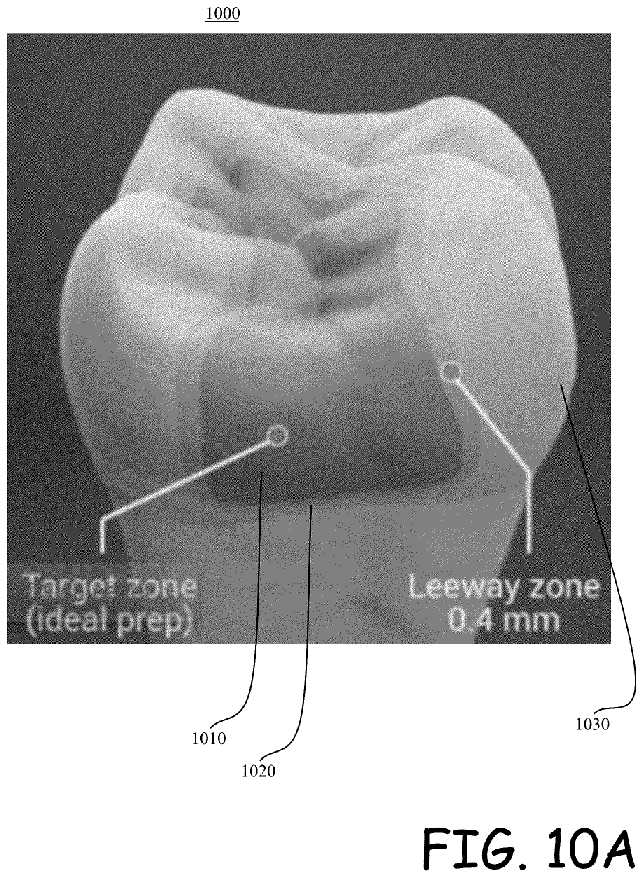

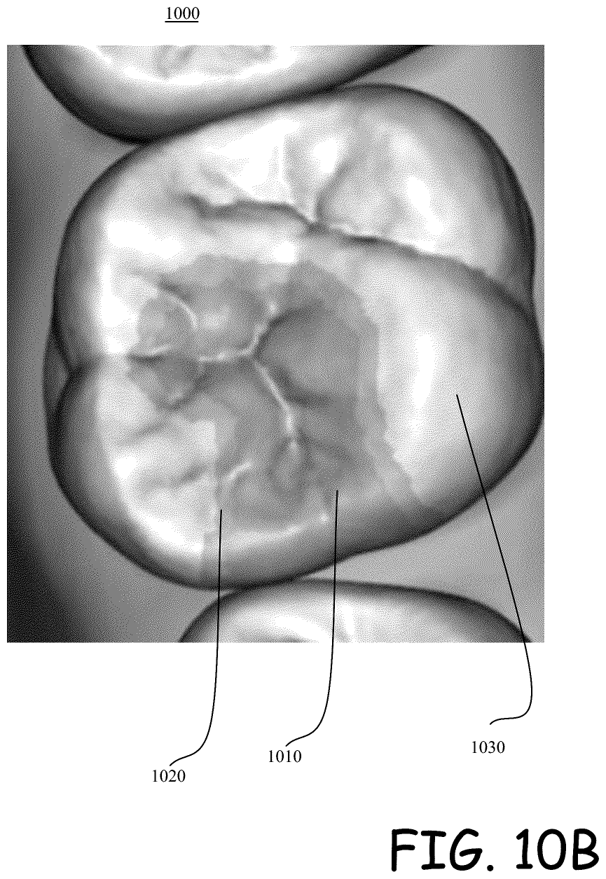

FIG. 10A illustrates an actual three-dimensional assessment tool deployable in an operational system. FIG. 10B illustrates a top-plan view of the assessment tool of FIG. 10A within the environment of a gum or tissue. In the illustrated embodiment, the assessment tool (or tooth) 1000 is made available to a user. In some embodiments, the assessment tool is an electronic model that is displayed on a screen such as the screen 152 of the haptic subsystem 150 illustrated in FIG. 1. In such embodiments, the assessment tool 1000 can be loaded and presented on the display and the user can utilize the haptic system 150 to perform work on the assessment tool. In other embodiments, the assessment tool 1000 may be a physical three-dimensional model that can be incorporated into a system similar to the system depicted in FIG. 1, or can be worked on by an actual live system and once work is completed on the assessment tool 1000, the resulting preparation can be scanned by scanner 120 and loaded into system 100 for comparing and evaluation of the performance.

The assessment tool 1000 in the illustrated embodiment is a tooth 1030 that is shown as being constructed of material (digitized or physical) that is presented in different colors. The area of the tooth 1030 that is not to be modified is illustrated in FIG. 10A and FIG. 10B as predominantly white in color. The tooth 1030 also includes a target zone 1010 that is presented in a second color. As illustrated in previous figures, while only a surface of the tooth 1030 is being illustrated, the target zone 1010, extends down below the surface of the tooth 1030 and basically represents a volume of the tooth 1030 that is to be removed. The purpose of the assessment tool 1000 is to present a task to the user (i.e. remove the material in the target zone 1010) and then provide immediate visual feedback as well as scoring the performance of the task based on a full comparison of the digital or physical result of the performance of the task. A variety of tasks can be set up using different configurations of the assessment tool 1000. For instance, the assessment tool 1000 can be configured to practice skills on removing matter for a cavity located on the top of the tooth, the front or back of the tooth, the side of a tooth or even a portion that is difficult to access due to being partially blocked by an adjacent tooth. Thus, the assessment tool 1000 can be utilized to not just test and develop proficiency in one procedure, but rather a wide range of different procedures.

The illustrated tooth 1030 also includes a leeway zone 1020 that is defined within the assessment tool 1000 and is basically a zone surrounding the target zone 1010. The leeway zone 1020 is shown in a different color from the target zone 1010 and the tooth 1030. The goal of the operator is to remove as much of the target zone 1010 as possible without removing any, or at least minimizing the amount, of the leeway zone 1020. If the operator removes too much material, the color of the tooth 1030 will be seen through the leeway zone 1020.

Where the assessment tool 1000 is a physical three-dimensional tooth, the operator attempts to physically remove the target volume 1010 from the tooth 1030, leaving as much of the leeway zone 1020 as the operator can. Once completed, the scanner 120 can be used to scan an image of the prepared assessment tool 1000 into the system 100. Prior to commencing work on the assessment tool 1000, the original, unmodified assessment tool can be scanned into the system 100. Once the modified assessment tool 1000 is scanned into system 100, the modified assessment tool 1000 can be compared to a model preparation that is within the database 180, obtained from a 3^{rd} party system 170 or created using the haptic system 150 on a scan of the unmodified assessment tool or some other digital representation of the same.

The compare sub-system 130 may then generate performance results and display the results for the operator (i.e., student and/or instructor). The performance results may be a score that is based on the percentage of over removal, under removal, (and in some cases over restoration and under restoration) in view of the amount of material that was intended to be removed (and/or restored). The score can take into consideration pre-programmed tolerances. For instance, for a beginner or novice, a larger tolerance may be imposed and for an advanced student or practitioner, a smaller tolerance may be imposed.

Thus, it can be appreciated that the various embodiments of the Dental System create a new, novel and unique system and method for identifying the performance of psychomotor skills. The system can be used for a variety of applications including dental school admittance, exercise and development of psychomotor skills, licensure examinations, recertification of practitioners, dental school curriculum, etc.

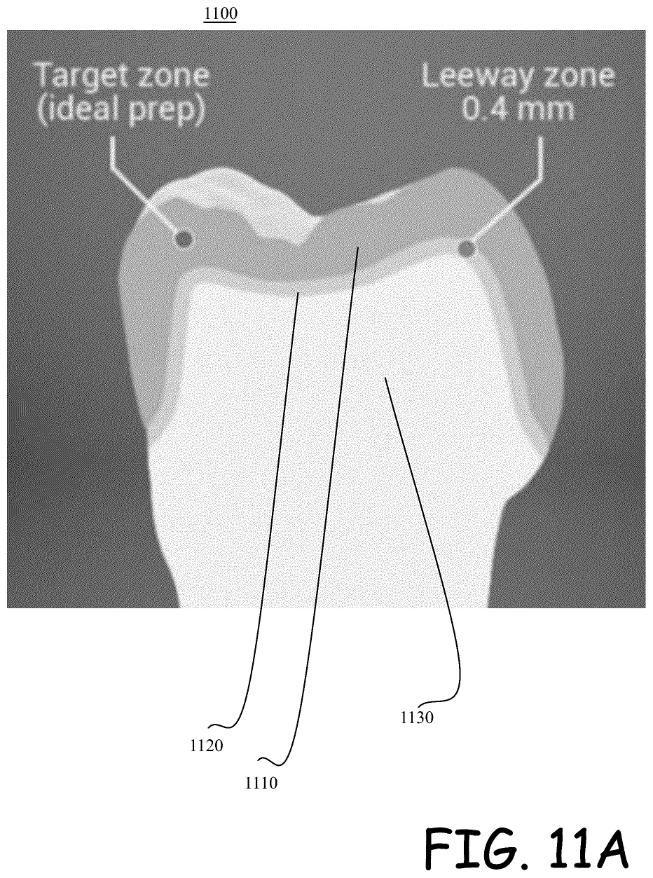

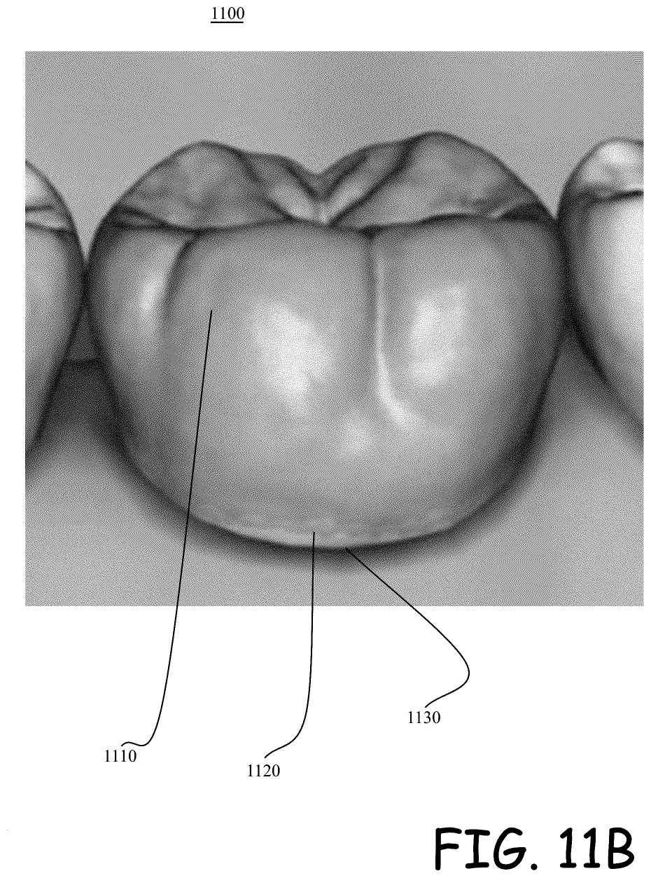

FIG. 11A illustrates an actual three-dimensional assessment tool deployable in an operational system. FIG. 11B illustrates a top-plan view of the assessment tool of FIG. 11A within the environment of a gum or tissue. In the illustrated embodiment, the assessment tool (or tooth) 1100 is made available to a user. In some embodiments, the assessment tool is an electronic model that is displayed on a screen such as the screen 152 of the haptic subsystem 150 illustrated in FIG. 1. In such embodiments, the assessment tool 1100 can be loaded and presented on the display and the user can utilize the haptic system 150 to perform work on the assessment tool. In other embodiments, the assessment tool 1100 may be a physical three-dimensional model that can be incorporated into a system similar to the system depicted in FIG. 1, or can be worked on by an actual live system and once work is completed on the assessment tool 1100, the resulting preparation can be scanned by scanner 120 and loaded into system 100 for comparing and evaluation of the performance.

The assessment tool 1100 in the illustrated embodiment is a tooth 1130 that is shown as being constructed of material (digitized or physical) that is presented in different colors. The area of the tooth 1130 that is to not be modified is illustrated in FIG. 11A and FIG. 11B as predominantly white in color. The tooth 1130 also includes a target zone 1110 that is presented in a second color. As illustrated in previous figures, while only a surface of the tooth 1130 is being illustrated, the target zone 1110, extends down below the surface of the tooth 1130 and basically represents a volume of the tooth 1130 that is to be removed. The purpose of the assessment tool 1100 is to present a task to the user (i.e., remove the material in the target zone 1110) and then provide immediate visual feedback as well as scoring the performance of the task based on a full comparison of the digital or physical result of the performance of the task. The illustrated tooth 1130 also includes a leeway zone 1120 that is defined within the assessment tool 1100 and is basically a zone surrounding the target zone 1110. The leeway zone 1120 is shown in a different color from the target zone 1110 and the tooth 1130. The goal of the operator is to remove as much of the target zone 1110 as possible without removing any, or at least minimizing the amount, of the leeway zone 1120. If the operator removes too much material, the color of the tooth 1130 will be seen through the leeway zone 1120.

Where the assessment tool 1100 is a physical three-dimensional tooth, the operator attempts to physically remove the target volume 1110 from the tooth 1130, leaving as much of the leeway zone 1120 as the operator can. Once completed, the scanner 120 can be used to scan an image of the prepared assessment tool 1100 into the system 100. Prior to commencing work on the assessment tool 1100, the original, unmodified assessment tool can be scanned into the system 100. Once the modified assessment tool 1100 is scanned into system 100, the modified assessment tool 1000 can be compared to a model preparation that is within the database 180, obtained from a 3^{rd} party system 170 or created using the haptic system 150 on a scan of the unmodified assessment tool or some other digital representation of the same.

The compare sub-system 130 may then generate performance results and display the results for the operator (i.e., student and/or instructor). The performance results may be a score that is based on the percentage of over removal, under removal, (and in some cases over restoration and under restoration) in view of the amount of material that was intended to be removed (and/or restored). The score can take into consideration pre-programmed tolerances. For instance, for a beginner or novice, a larger tolerance may be imposed and for an advanced student or practitioner, a smaller tolerance may be imposed.

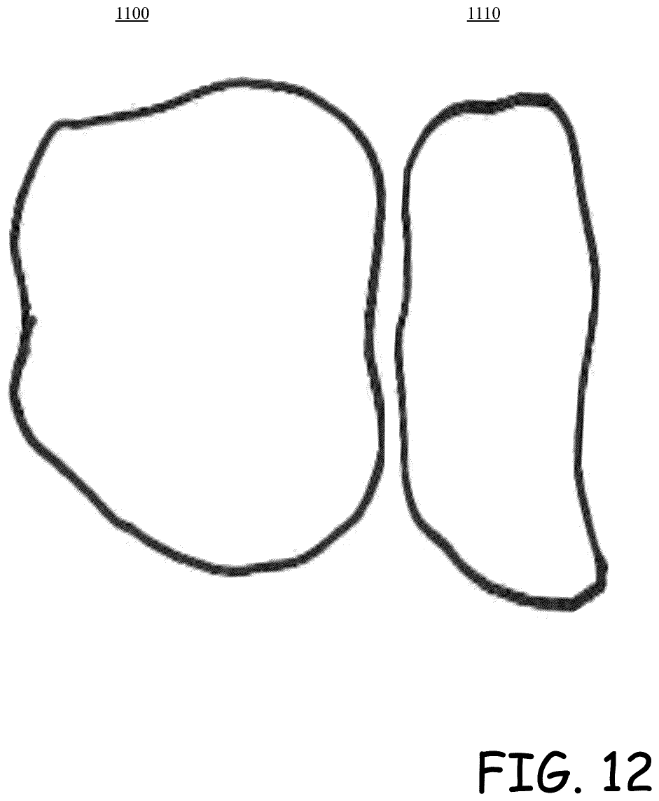

FIG. 12 is yet another exemplary embodiment of a target model system on which a student or practitioner can strive to work. For example, the embodiment of the assessment tool in FIGS. 7A, 7B and 7C may be utilized for an individual to begin developing psychomotor skills for a particular procedure. As the individual's skills develop, the individual may move to the assessment tool illustrated in FIG. 8. Likewise, with more experience, the individual can move to the assessment tool illustrated in FIG. 9 and the ultimate goal, prior to working on actual live subjects, is to master the procedure on the model illustrated in FIG. 12. In this model, tooth 1200 and 1210 give the appearance of an actual tooth for the student to work with without any color guidance. In this scenario the student's efforts can be compared to an ideal model to determine if the student has removed the appropriate amounts of the tooth.

The use of scoring with regards to the assessment tools has been described from the perspective of identifying the percentage of the colored portions that are removed or that remain. In addition, scoring can also be applied in real-time using the system as presented in FIG. 1. The screen (151) associated with haptic subsystem (150) displays both the color representation of the assessment as well as a numerical percentage of the colored portions. This combined assessment is done in real-time, thus allowing the user to have both physical visual feedback and computer analyzed feedback.

It will thus be appreciated that one embodiment includes an apparatus for training and assessing dental procedure skills. The apparatus includes a structure consisting of a material in a first color that is suitable to be processed using one or more dental tools; a target zone consisting of a material in a second color that is suitable to be processed using one or more dental tools; and a boundary zone consisting of a material in a third color that is suitable to be processed using one or more dental tools. In operation, the target zone is material that is to be removed from the apparatus during a procedure and the boundary zone is material that is not to be removed from the apparatus during the procedure, whereby the apparatus can be visually inspected to determine the accuracy of the performance of the procedure.

It should also be appreciated that rather than or in addition to a physical indictor, the various layers of material may have certain characteristics that enable other forms of detection by means of electronic scanning or the like. Thus, a scanner would be able to take a snap-shat of the item and by differentiating the material, the scanner can determine how well the procedure was performed. Materials that could be utilized include conductive and non-conductive materials, materials with different densities, materials with different heat absorption characteristics, etc.

It should be appreciated that the various embodiments provide not only a physical indicator to the student or practitioner, but also, within the Dental System, the practice preparation can be compared to an ideal preparation to generate a score for the student or practitioner. The compare sub-system 130 is equipped to identify any green material that has inadvertently been removed from the block or tooth, as well as any red material that was not properly removed. Each under removal and over removal reduces the overall points or score attributed for the exercise.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art.

It will be appreciated by persons skilled in the art that the present invention is not limited by what has been particularly shown and described herein above. Rather the scope of the invention is defined by the claims that follow.

What is claimed is:

1. An apparatus for training and assessing dental procedure skills, the apparatus comprising:
a model consisting of a material in a first color that is suitable to be processed using one or more dental tools;
a target zone in the model consisting of a material in a second color that is suitable to be processed using one or more dental tools;
a boundary zone in the model consisting of a material in a third color that is suitable to be processed using one or more dental tools; and
wherein, the target zone identifies material that is to be removed from the apparatus during a procedure and the boundary zone is material that is not to be removed from the apparatus during the procedure, whereby the apparatus can be visually inspected to determine the accuracy of the performance of the procedure.

2. The apparatus of claim 1, wherein the apparatus comprises a breech zone consisting of material in a color that is visually distinguished from the second color and the third color and is suitable to be processed using one or more dental tools, wherein the breach zone is visible when too much boundary zone material has been removed.

3. The apparatus of claim 1, wherein the apparatus is a three-dimensional representation of a tooth.

4. The apparatus of claim 3, wherein the apparatus is a physical element and utilizing the one or more dental tools to physically remove matter from the physical element, a user is able to create a user preparation.

5. The apparatus of claim 4, wherein the user preparation can be scanned into a processing system and compared to an ideal preparation.

6. The apparatus of claim 3, wherein the apparatus is a digital representation that is displayed on a display of a haptic dental system and the one or more dental tools are physical tools that are communicatively coupled to the haptic dental system and provide haptic feedback to a user as the user virtually removes matter from the digital representation.

7. The apparatus of claim 6, wherein the digital representation provides real-time feedback on the display with respect to the matter that has been virtually removed from the apparatus.

8. The apparatus of claim 6, wherein the digital representation that has matter virtually removed is a virtual user preparation.

9. The apparatus of claim 8, wherein the virtual user preparation can be compared within the haptic dental system to an ideal preparation to obtain a real-time score.

10. A dental system for training and assessing dental procedure skills, the system using an apparatus and comprising:
a dental tool that processes an apparatus;
the apparatus comprising:
a model consisting of a material in a first color that is suitable to be processed using one or more dental tools;
a target zone in the model consisting of a material in a second color that is suitable to be processed using one or more dental tools;
a boundary zone in the model consisting of a material in a third color that is suitable to be processed using one or more dental tools; and
wherein, the target zone identifies material that is to be removed from the apparatus, using the dental tool during a procedure and the boundary zone is material that is not to be removed from the apparatus during the procedure, whereby the apparatus can be visually inspected to determine the accuracy of the performance of the procedure.

11. The dental system of claim 10, wherein the apparatus further comprises a breech zone consisting of material in a color that is visually distinguished from the second color and the third color and is suitable to be processed using one or more dental tools, wherein the breach zone is visible when too much boundary zone material has been removed.

12. The dental system of claim 10, wherein the apparatus is a three-dimensional representation of a tooth.

13. The dental system of claim 12, wherein the apparatus is a physical element and utilizing the dental tool to physically remove matter from the physical element, a user is able to create a user preparation.

14. The dental system of claim 13, wherein the user preparation can be scanned into the system and compared to an ideal preparation.

15. The dental system of claim 12, wherein the dental system is a haptic dental system and the apparatus is a digital representation that is displayed on a display of the haptic dental system and the dental tool is a physical tool that is communicatively coupled to the haptic dental system and provides haptic feedback to a user as the user virtually removes matter from the digital representation.

16. The haptic dental system of claim 15, wherein the digital representation provides real-time feedback on the display with respect to the matter that has been virtually removed from the apparatus.

17. The haptic dental system of claim 15, wherein the digital representation that has matter virtually removed is a virtual user preparation.

18. The haptic dental system of claim 17, further comprising a compare sub-system that identifies the differences between the virtual user preparation and an ideal preparation and identifies a real-time score.
